(19) United States
(12) Patent Application Publication
(10) Pub. No.: US 2006/0018952 A1
(43) Pub. Date: Jan. 26, 2006

(54) Molecular reactive chitosan finishing agent used for textile

(76) Inventor: Chen Liang, Shanghai (CN)

Correspondence Address: EDWARDS & ANGELL, LLP, P.O. BOX 55874, BOSTON, MA, 02205, US

(21) Appl. No.: 11/013,895

(22) Filed: Dec. 15, 2004

(30) Foreign Application Priority Data
Jul. 22, 2004 (CN) 200410053085.6

(51) Int. Cl. A61K 9/70; C08B 37/08

(52) U.S. Cl. 424/443; 536/20

(57) ABSTRACT

A method for making a molecular chitosan finishing agent used for textile by adding an amino-alcohol derivative and an anionic surfactant into a chitosan acetic acid with different solubilities.

### TECHNICAL FIELD

The technology involves biological-chemical technical fields, namely, it is a molecular reactive chitosan finishing agent used for textile.

### TECHNICAL BACKGROUND

Chitosan is grafted into the textile to make the textile antibacterial, fungusproof, deodorant and antistatic. The present technology has two disadvantages: 1. chitosan is dissolved in weak acid, and then is coated on the textile, however, the chitosan can gradually hydrolyze in acid solution until it turns into monosaccharose and loses all biological activities; 2. the chitosan in the solution is homomolecular, and as for different bacteria (gram-positive bacteria and gram-negative bacteria) and fungus, adjusting the molecular weights of chitosan can get the best effects.

Content

The invention uses biological-chemical methods to respectively add an amino-alcohol derivative and an anionic surfactant into chitosan acetic acid solution with different solubilities and control the reaction temperature and time. Therefore, it effectively controls the hydrolysis degree and the molecular weight of chitosan which makes its functions lasting and stable, and gain a mixing liquid of chitosan with different molecular weights. The mixing liquid can inhibit different kinds of bacteria and fungus.

To meet above purposes, the features of the molecular reactive chitosan finishing agent designed are to respectively add an amino-alcohol derivative and an anionic surfactant into chitosan acetic acid solution with different solubilities, strictly control the reaction temperature and time, stir several times, stew, and finally age.

Compared with the present technology, the invention can effectively control the hydrolysis degree and molecular weight of chitosan, and can gain a mixing liquid of chitosan with different molecular weights, which make the properties of the products stable, reliable and lasting with wide application, high biological activities, simple technological process, small investment and no pollution and can be used in industrial production.

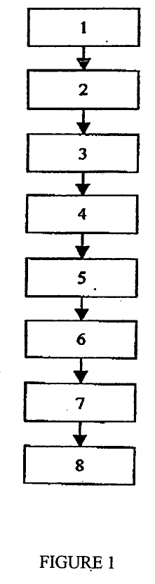

### ILLUSTRATION OF THE FIG. 1

FIG. 1 shows the technological process of the invention.

See FIG. 1

1: raw material of chitosan;

2: dissolving in acetic acid solution

3: adding amino-alcohol derivative and an anionic surfactant

4: mixing and stirring

5: filtrating;

6: stewing;

7: aging

8: product.

### EXAMPLES

The following is the detailed description of the invention by FIG. 1. The technology is clear for the professionals.

The raw material of chitosan is equally divided into three parts which are respectively added into acetic acid solution. The solution temperatures are respectively 70-80° C., 40-50° C. and 20-25° C., and the time is respectively 5-10 h, 45-50 h and 95-100 h. Afterwards, amino-alcohol derivative and an anionic surfactant are respectively added. Then, three batch chitosan acetic acid solutions are mixed, stirred fully, filtrated and stewed for 20-25 h, aged for 40-50 h in incubator. Finally, chitiosan finishing agent is produced.

1. The features of the molecular reactive chitosan finishing agent are that amino-alcohol derivative and anionic surfactant are added into chitosan acetic acid solution.

2. Method of claim 1 wherein said the weight percent of amino-alcohol derivative is 3-10%.

3. Method of claim 1 wherein said the weight percent of the anionic surfactant is 2-4%.

4. The process of the molecular reactive chitosan finishing agent comprises the steps of:
a) equally dividing the total chitosan into three parts which are respectively added into acetic acid solution at different temperatures and dissolved in different time;
b) respectively adding amino-alcohol derivative and anionic surfactant;
c) mixing, stirring, filtrating, stewing, aging which produce the molecular reactive chitosan finishing agent.

5. Method of claim 1 wherein said the dissolving time is 9-10 h, 45-50 h and 95-100 h, and the temperatures are 70-80° C., 40-50° C. and 20-25° C.

6. Method of claim 1 wherein said the stewing time is 20-25 h and the temperature ranges between 20° C. and 25° C.

7. Method of claim 1 wherein said the aging time is 40-50 h and the temperature ranges between 20° C. and 25° C.

8. Method of claim 1 wherein said the molecular reactive chitosan finishing agent can be used in the textile finishing field such as textile fibers, textile face fabric and textile products.

9. Method of claim 4 wherein said the dissolving time is 9-10 h, 45-50 h and 95-100 h, and the temperatures are 70-80° C., 40-50° C. and 20-25° C.

10. Method of claim 4 wherein said the stewing time is 20-25 h and the temperature ranges between 20° C. and 25° C.

11. Method of claim 4 wherein said the aging time is 40-50 h and the temperature ranges between 20° C. and 25° C.
